(19) United States
(12) Patent Application Publication
(10) Pub. No.: US 2016/0007639 A1
(43) Pub. Date: Jan. 14, 2016

(54) FRUIT AND VEGETABLE-BASED FORMULATIONS AND METHODS AND USES THEREOF

(71) Applicant: BlendFresh, LLC, Orem, UT (US)

(72) Inventors: Jake Smith, Pleasant Grove, UT (US); Jeff Wasden, Springville, UT (US); Christine Beltran, Salem, UT (US)

(21) Appl. No.: 14/329,755

(22) Filed: Jul. 11, 2014

(51) Int. Cl. A23L 1/212; A61Q 19/00; A61Q 5/02; A23L 1/216; A61Q 1/00; A61Q 19/10; A23K 1/14; A61K 8/97; A61Q 5/12

(52) U.S. Cl. CPC A23L 1/212; A61K 8/97; A61Q 19/00; A61Q 5/02; A61Q 5/12; A61Q 1/00; A61Q 19/10; A23K 1/143; A23K 1/14; A23L 1/216; A61K 2800/592; A61K 2800/30; A23V 2002/00

(57) ABSTRACT

This application concerns compositions including at least one whole or partial food fruit component and/or at least one whole or partial food vegetable component. Also, methods for preparing a fruit and vegetable-based food product formulation for animal or human consumption including blending said composition and a liquid in a blender to form a slurry. Further, uses of said composition as a nutritional source or supplement for humans or animal.

Throughout this disclosure, several publications (including, without limitation, patents) are referenced herein. These publications are incorporated in their entireties by this reference.

### FIELD OF THE INVENTION

The present invention relates generally to the field of nutrition and in particular food product formulations based on fruit and/or vegetables for animal or human consumption, and methods and uses thereof. More particularly, the present invention relates to novel compositional blends of whole or partial food fruit and vegetables combined by pigment and/or phytonutrients compositions.

### BACKGROUND OF THE INVENTION

The majority of Americans do not consume the recommended quantity of fruits and vegetables, according to the Centers for Disease Control. Consuming an adequate amount and variety of colorful raw whole foods has been shown to mitigate and prevent disease. The guidelines contained in the Centers for Disease Control booklet titled “Strategies to Prevent Obesity and Other Chronic Diseases” recommends five servings daily of fruits and vegetables. However, over 90% of American adults eat less than 1 fruit or vegetable a day. To achieve optimal health, plant foods should comprise 80% of dietary intake.

Additionally, the colors associated with each fruit or vegetable have a specific purpose for the animal or human body. Each fruit or vegetable includes naturally occurring phytochemicals and/or pigments which each have its own mechanism of action and function. For example, mangiferin which has a distinctive gold-orange fluorescent color is naturally occurring in mangoes and has shown to exert antitumor activity in breast cancer cells. See, e.g., Li, et al, “Mangiferin exerts antitumor activity in breast cancer cells by regulating matrix metalloproteinases, epithelial to mesenchymal transition, and β-catenin signaling pathway”, Toxicol. Appl. Pharmacol., 2013 Oct. 1; 272(1):189-90, doi: 10.1016/j.taap.2013.05.011. Epub 2013 May 22.

Additionally, beta-carotene, a precursor to Vitamin A, is an essential nutrient that makes carrots orange, and has demonstrated to correct myopathy, improve visual acuity and prevent age related acuity and focus. Other nutrients include lycopene (which gives the red/pink color in tomatoes), lutein (related to greens), resveratrol (found in grapes), ellagic acid, kaempferol, catechins, tiliroside, ferulic acid, caffeic acid terpenoids, indole, and many others. In terms of variety, it is recommended that persons should eat at least three different colors of fruits and vegetables daily to get the phytonutrients found in colorful fruits and vegetables. Colorful fruits and vegetables are the key feature of any diet in providing disease-lighting compounds. Additionally, it is recommended that each person should consume at least 3 servings per week of vegetable protein in the form of legumes, nuts, or seeds.

Consumption of fruits and vegetables has also been shown to mitigate or ameliorate the risks of more serious health conditions. For example, in a recent prospective cohort study of 41,837 postmenopausal women, the association of fruit and vegetable consumption with lung cancer risk was investigated. Researchers found that the risk of lung cancer was cut in half when the consumption of fruits and vegetables increased from 24 or less servings to an excess of 48 servings per week. Similarly, the risk of lung cancer was also cut in half when the consumption of green leafy vegetables, including spinach and parsley sources, increased from 1 or fewer servings to 6 or more servings per week. Steinmetz, K. et al., “Vegetables, Fruit, and Lung Cancer in the Iowa Women's Health Study,” Cancer Res. 53:536-43 (1993). Another study found that an increased intake of fresh tomatoes (a major source of lycopene) was associated with a pattern of projection for all sites of digestive tract cancer. Stahl, W. et al., “Lycopene: A Biologically Important Carotenoid for Humans?” Arc. Biochem. Biophys. 336:1-9 (1996).

Additionally, studies have also shown that diets high in plant foods are associated with reduced risk of various types of cancers. These studies correlate diets rich in fruits and vegetables and nutrients from these diets, such as vitamin C, vitamin A, and dietary fiber, with reduced cancer risk. Persons consuming these diets frequently have high intakes of these nutrients.

There are many barriers that prevent consumption of the recommended number of fruits and vegetables. Geographically, there are fruit and vegetable-deprived areas where people live who are not adequately nourished or do not have access to colorful fruits and vegetables that are stable and still retain their color, potency and nutrient density. Further, fruits and vegetables spoil quickly because they often have a high water content allowing mold, bacteria and fungus to grow rapidly. Finally, many households do not use up the fruits and vegetables they buy. Once picked or prepared, many consumers are unable to use up the entirety of a fruit or vegetable because of the short shelf life of the produce.

There regains a need, therefore, for convenient vehicles for raw whole or partial fruits and veggies, which have potent essential nutrients, pigments and phytonutrients high in biomedically active plant nutrients, but that are stable and have a lengthy shelf-life.

### BRIEF SUMMARY OF THE INVENTION

The present invention is based on the discovery of compositions of whole or partial food fruits and/or vegetables combined by pigment and/or phytonutrients, and developed to address the problems with existing delivery of fruits and vegetables outlined above.

In one aspect, the present invention provides compositions comprising at least one whole or partial food fruit component and at least one whole food vegetable component, wherein both the at least one whole or partial food fruit component and the at least one whole or partial food vegetable component which have the same or similar pigment group selected from the group consisting of red, yellow/orange, purple/blue, green, black, and white. The at least one whole or partial food fruit component and the at least one whole or partial food vegetable component can be juiced or pureed, combined, and then dehydrated using Fresh Dried™ technology or any drying/dehydrating technology presently known in the art or discussed below.

In another aspect, the composition can comprise at least 10 separate whole or partial fruit or vegetable components, or, in further aspects, at least 12 separate whole or partial fruit or vegetable components. The drying/dehydration can, in some embodiments, be achieved using rapid zone drying, window refractance, vacuum belt drying, or other low-temperature drying.

In another aspect, phytonutrients can, in further aspects, be present in the amount of from 0.1 meg to 20,000 mg/day or from 1000 meg to 20,000 mg/day from each source. Phytonutrients from green pigment group components include, but not limited to, chlorophyll, lutein, glutathione, alpha lipoic acid, D-glucaric acid, saponins, patuletin, spinacetin, spinatoside, rutin, or lignans. Phytonutrients from red pigment group components include, but not limited to, lycopene, zeta carotene, kaempferol, quercetin, p-coumaric acid, delphyinidin, malvidin, cyanidin, chlorogenic acid, phytoene, phytofluene, epigallocatechin gallate, ellagic acid, benzoic acid, lignans, myricetin, ferulic acid. Phytonutrients from yellow/orange pigment group components include, but not limited to, hesperetin, limonin, nomilin, d-limonene, beta-sitosterol, mangiferine, syringic acid, beta cryptoxanthin, ferulic acid, chlorogenic acid, or vanillic acid. Phytonutrients from purple/blue pigment group components include, but not limited to, cyaniding, delphinidin, malvidin, peonidin, petunidin, catechins, geraniol, resveratrol, tartaric acid, quercitin, tannins, saponins, caffeic acid, chloregenic acid, sorbitol, or malic acid.

In another aspect, the at least one whole or partial food fruit component and at least one whole or partial food vegetable component each have green pigmentation. In such cases, the at least one whole or partial food fruit component and the at least one whole or partial food vegetable component are selected from the group consisting of green pea, grapes, honeydew melon, pear, artichoke, green apple, green beans, cabbage, okra, watercress, peppers, broccoli, spinach, kale, kiwi, asparagus, avocado, collards, lime, romaine, brussels sprouts, algae, spirulina, fennel, cucumber, zucchini, and/or Swiss chard.

In a further aspect, the at least one whole or partial food fruit component and the at least one whole or partial food vegetable component each have red pigmentation. In such cases, the at least one whole or partial food fruit component and at least one whole or partial food vegetable component are selected from the group consisting of beets, apples, strawberries, red pear, raspberries, cranberries, blood oranges, red grapes, pomegranate, cherry, peppers, rhubarb, prickly pear, watermelon, cabbage, radishes, radicchio, red onion, red potato, grapefruit, crampberry, and/or tomato.

In a further aspect, the at least one whole or partial food fruit component and the at least one whole or partial food vegetable component each have yellow/orange pigmentation. In such cases, the at least one whole or partial food fruit component and the at least one whole or partial food vegetable component are selected from the group consisting of carrot, butternut squash, sweet potato, mango, pineapple, pumpkin, apricot, peach, banana, beets, figs, watermelon, potato, persimmons, yellow bell peppers, lemon, cantaloupe, papaya, star fruit, grapefruit, tangerine, and/or orange.

In a further aspect, the at least one whole or partial food fruit component and the at least one whole or partial food vegetable component each have blue/purple pigmentation. In such cases, the at least one whole or partial food fruit component and the at least one whole or partial food vegetable component are selected from the group consisting of purple beets, purple cabbage, concord grape, acai, blackberry, blueberry, elderberries, figs, kohlrabi, figs, peppers, potato, huckleberry, chokecherry, mulberry, plum, purple carrot, corn, cauliflower, poi, black currant, eggplant, black raspberry, purple carrot, acerola, cauliflower, potato, and/or passion fruit.

In a further aspect, the at least one whole or partial food fruit component and the at least one whole or partial food vegetable component each have white pigmentation. In such cases, the at least one whole or partial food fruit component and the at least one whole or partial food vegetable component are selected from the group consisting of crimini mushroom, lychee, rambutan, Fijian longan, dragon fruit, white peach, white pear, white nectarine, parsnip, shallots, jicama, Jerusalem artichoke, onion, leeks, cauliflower, and/or coconut.

In a further aspect, the at least one whole or partial food fruit component and the at least one whole or partial food vegetable component each have black pigmentation. In such cases, the at least one whole or partial food fruit component and the at least one whole or partial food vegetable component are selected from the group consisting of black olives, black cherries, black plums, black currants, and/or black figs.

In another aspect, the composition can have a decreased likelihood of clumping or hardening in a sealed package than a single-component composition. The compositions can be used for animal or human consumption in a smoothie, fruit drink, shake, food coloring, flavoring, baby food, sports supplemental product, baked good, or other food product. In another aspect, the composition can be used in a product for topical use that is applied to body surfaces (e.g., skin or mucous membrane) or hair, such as, but not limited to, a lotion, cream, soap, gel, foam, ear drops, eye drops, inhalers (e.g., dry powder inhalers, metered-dose inhalers, nebulizers), mist and ointment. In another aspect, the composition can be used in a product for sun protection, including but not limited to sunscreen lotion or spray. In still further aspect, the compositions can further comprise additional ingredients such as herbs, spices, vitamins, minerals, food additives for preservation of and/or improved taste, freshness, or mouthfeel, or a combination thereof.

In a further aspect, wherein the at least one whole or partial food fruit component and the at least one whole or partial food vegetable component each have a 1-99 weight percent of the composition.

In a bather aspect, the composition is formulated as a powder, flakes, tablet, or capsule.

In another aspect, use of the composition as a nutritional source or supplement for humans.

In another aspect, a method for preparing a fruit and vegetable-based food product formulation for animal or human consumption comprising: blending the composition and a liquid (e.g., water) in a high-speed blender to form a slurry.

### DETAILED DESCRIPTION OF THE INVENTION

The illustrative embodiments herein described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and made part of this disclosure.

The present invention is based upon the discovery of dried fruit and/or vegetable compositions that can provide a stable source of fruits, vegetables, and necessary phytonutrients with a long shelf-life, decreased clumping and hardening, and health benefits not decreased from the fresh forms of the fruits and vegetables. Also, such compositions are formulated in a manner that promotes convenience of use and improved cellular absorption. Further, no artificial flavors, preservatives, colors, binders, or fillers are added into the compositions of the present invention since the preparation process discussed herein (e.g., drying/dehydration techniques) preserves a high amount of phytonutrients, pigments, and other health-enriching characteristics of each fruit or vegetable used in the compositions discussed herein.

### Fruits and Vegetables

Fruits and vegetables must be harvested at the time of ripening, crispness, freshness, taste, appearance, condition, and highest nutritive value. Because of the perishable nature of fruits and vegetables, harvesting and handling speed is of utmost importance. Any techniques known to those in the art may be used to extend postharvest shelf life, including removal from direct sunlight and reducing the respiration by hydro or rapid cooling; slow respiration by maintaining optimal shipping and storing environment; or maintaining optimal relative humidity. Special skills are required for proper harvesting, handling, grading to ensure optimum quality. Fruits an vegetables are cleaned, then prepared, by removing stems, leafs and other inedible parts. Additional measures are taken to ensure high quality, premium fruit and vegetable.

The fruits and vegetables used in the compositions described herein are grouped by phytonutrients or pigmentation. Generally, fruits and vegetables having a red pigmentation can be grouped together; fruits and vegetables having a yellow/orange pigmentation can be grouped together; fruits and vegetables having a green pigmentation can be grouped together; fruits and vegetables having a purple/blue pigmentation can be grouped together; fruits and vegetables having a white pigmentation, can be grouped together; and fruits and vegetables having a black pigmentation can be grouped together. The fruits and vegetables can be grouped regardless of whether they share or have the same pigmentation. For example, fruits and vegetables can be grouped by one or more phytonutrients, regardless of pigmentation.

Each whole or partial fruit or vegetable component can be provided in any concentration. For example, each component can be about 1% by weight, about 2% by weight, about 4% by weight, about 5% by weight, about 10% by weight, about 15% by weight, about 18% by weight, about 20% by weight, about 22% by weight, about 23% by weight, about 24% by weight, about 25% by weight, about 30% by weight, about 35% by weight, about 40% by weight, about 45% by weight, about 50% by weight, about 55% by weight, about 60% by weight, about 70% by weight, about 80% by weight, about 90% by weight, or about 99% by weight. Also, each component can have a range (by weight) of 1%-2%; 2%-4%; 1%-5%; 2%-4%; 2%-5%; 4%-6%; 5%:7%; 6%-8%; 7%-10%; 8%-11%; 8%-14%; 9%-14%; 12%-14%; 15%-19%; 15%-22%; 15%-24%; 19%-21%; 19%-24%; 21%-24%; 24%-33%; 25%-30%; 1%-15%; 1%-18%; 1%-20%; 1%-22%; 1%-23%; 1%-24%; 1%-25%; 1%-30%; or 30%-99%.

Pigmentation compositions can include a minimum of 12 fruits sod vegetables components which provide phytonutrients in certain ratios and levels present in a portion ranges of approximately 0.1 meg to 20,000 mg/day or 1000 meg to 20,000 mg/day from each red pigment source including but not limited to beets, apples, strawberries, red pear, raspberries, cranberries, pomegranate, acerola cherry, red peppers, rhubarb, prickly pear, and tomato. Purple/Blue pigments include but not limited to fruits and/or vegetables which provide phytonutrients in certain ratios and levels present in a portion ranges of approximately 0.1 meg to 20,000 mg/day from purple beets, purple cabbage, concord grape, acai, blackberry, blueberry, plum, purple carrot, black currant, eggplant, black raspberry, and passion fruit. Yellow/orange pigment sources include but not limited to fruits and/or vegetables which provide phytonutrients in certain ratios and levels present in a portion ranges of approximately 0.1 meg to 20,000 mg/day from carrot, butternut squash, sweet potato, mango, pineapple, pumpkin, apricot, peach, banana, yellow bell peppers, lemon and orange. Green pigment sources include but not limited to fruits and/or vegetables, which provide phytonutrients in certain ratios and levels present in a portion ranges of approximately 0.1 meg to 20,000 mg/day from green pea, apple pulp, broccoli, spinach, kale, kiwi, asparagus, collards, lime, romaine, brussel sprouts, and Swiss chard.

The compositions described herein include ratios and levels of enzymes, organic substances, metabolites, phytonutrient families, terpenes, carotenoids, polyphenolics, anythocyanins, glycosides, polysaccharides, flavonoids, from various whole food sources including but not limited to: rasaceae, vitaceae, rutaceae, lythraceae, schisandraceae, cucrbitaceae, sapindaceae, rhammnaceae, malvaceae, elaeagnaceae, organized by fruits and vegetables by pigments or color from a minimum of 2, a minimum of 5, a minimum or 10, or a minimum of 12 sources or more of red, yellow/orange, purple/blue, green, white and black colored traits and vegetables. Such substances in these compositions have been shown to aid in the prevention and/or treatment of tumor growth, cancer formation, cancer diffusion, abnormal angiogenesis, neuroprotective disorder, diabetes, inflammation, atherosclerosis, viral infection, toxicity, glycemia, oxidation, leishmanial, molluscicidal, choleretic, spasmodic, immune and autoimmune disease, blood coagulation, osteoarthritis, rheumatoid arthritis, cataracts, liver malfunction, kidney disease, blood sugar disorders, fatigue, wound healing, and metabolic syndrome.

### Drying/Dehydration Techniques

The compositions described herein can be dried using any low-temperature drying mechanism known in the art. For example, the compositions can be dried using rapid zone drying, window reflectance, vacuum belt drying, or other low temperature drying. For example, the method of U.S. Pat. No. 4,631,837 can be used, which involves floating an infrared-transparent on the surface of a body of heated water and placing the material to be dried, such as the pulp and juice mix, on top of the film to absorb heat from the water and thereby cause drying/dehydration of the material. The apparatus for carrying out this drying method includes a reservoir of water, a film floated on the water, and a heater for maintaining the temperature of the water at a predetermined level. In a preferred aspect, the reservoir is in the form of an elongated trough and the film comprises an endless strip of polyester material, such as Mylar, to form a conveyor belt that floats on the surface of the water in the trough and carries the material to be dried on it.

In another example, the method of U.S. Pat. No. 6,539,645 can be used for drying/dehydrating of the compositions. The method involves placing the product on a first side of a support surface, and directing dry infrared light toward the second side of the surface to remove moisture from the product. A sensor can be included to measure at least one characteristic of the product, such as the temperature or moisture content thereof. The temperature of the light source can be regulated as a function of the measured characteristic. The support surface can also be made so as to be movable relative to the light source. In an alternative method, a plurality of control zones is defined and through which the product is successively passed. Each of the control zones has at least one associated light source and an associated sensor so as to regulate the temperature of the light sources associated with each control zone independently of those associated with another zone.

The compositions described herein can also include any other ingredients deemed desirable or known in the art, such as, but not limited to, the following: vitamins, minerals, antioxidants, spices, herbs, or other food additives for the preservation of taste, freshness, consistency or mouthfeel. Such additives are well known in the art.

Exemplary food fruit and vegetable components for red pigmented compositions, orange/yellow pigmented compositions, green pigmented compositions, and purple/blue pigmented compositions are described as follows in TABLE 1:

TABLE 1

| | Orange/Yellow | Green | Purple/Blue |
|---|---|---|---|
| Red-Pigmented | Pigmented | Pigmented | Pigmented |
| Sources | Sources | Sources | Sources |
| Apple | Carrot | Green Peas | Beets |
| Beets | Butternut Squash | Apple | Purple Cabbage |
| Red Cabbage | Sweet Potato | Broccoli | Concord Grape |
| Strawberry | Mango | Green Cabbage | Purple Carrot |
| Red Pear | Banana | Spinach | Acai |
| Raspberry | Pineapple | Kale | Blackberry |
| Cranberry | Pumpkin | Kiwi | Blueberry |
| Pomegranate | Apricot | Asparagus | Plum |
| Acerola Cherry | Peach | Collards | Acerola |
| Red Pepper | Yellow Bell Pepper | Acerola | Black Currant |
| Rhubarb | Lemon | Lime | Eggplant |
| Prickly Pear | Orange | Brussels Sprouts | Black Raspberry |
| Tomato | Beet | Swiss Chard | Passion Fruit |
| Red grapes | Fig | Romaine | Huckleberry |
| Cabbage | Watermelon | Avocado | Mulberry |
| Radishes | Potato | Fennel | Elderberries |
| Radicchio | Persimmon | Cucumber | Figs |
| Red onion | Star Fruit | Watercress | Kohlrabi |
| Crampberry | Tangerine | Okra | Poi |
| Red Grapefruit | Grapefruit | Pear | Cauliflower |
| Watermelon | | Sprulina | Potato |
| Blood Orange | | Melon (e.g., | Purple Carrot |
| | | Honeydew | |
| | | Melon) | |

The present invention will now be described in greater detail by reference to the following non-limiting examples.

### EXAMPLES

### Example 1

### Formulation of Dried Fruit and Vegetable Compositions

To formulate the dried fruit and vegetable compositions, fruits and vegetables are harvested at the time of ripening, crispness, freshness, taste, appearance, condition, and highest nutritive value. Because of the perishable nature of fruits and vegetables harvesting and handling speed is of utmost importance. Several techniques that are used to extend postharvest shelf life are known to those of skill in the art, and one or more of the following is employed: removal from direct sunlight and reducing the respiration by hydro or rapid cooling; slow respiration by maintaining optimal shipping and storing environment; and/or maintaining optimal relative humidity. Fruits and vegetables are cleaned, then prepared, by removing stems, leaves and other inedible parts.

Each individual fruit or vegetable is juiced or pureed, kept cool, and then mixed together with additional fruits and vegetables of the same pigment, according to the combinations found in TABLE 2 (green pigmented compositions), TABLE 3 (orange/yellow pigmented compositions), TABLE 4 (purple/blue pigmented compositions), and TABLE 5 (purple/blue pigmented compositions). Once the fruits and vegetables of the same or similar pigment have been mixed together they undergo fresh drying. There are only a few low temperature approved drying methods that can be used, such as rapid zone drying, window refractance, vacuum belt drying, or other low temperature drying. Once dried, each of the sources is tested.

TABLE 2
GREEN PIGMENTED COMPOSITIONS

| Component | Latin Binomial | Part Used | Weight % |
|---|---|---|---|
| Peas | Pisum sativum | Whole Vegetable | 1-24% |
| Apple | Malus domestica | Whole Fruit | 1-24% |
| Apple Juice | Malus domestica | Fruit Juice | 1-24% |
| Kiwi | Actinidia deliciosa | Whole fruit | 1-24% |
| Broccoli | Brassica oleracea | Whole Vegetable | 1-22% |
| Spinach | Spinacia oleracea | Leaf and steam juice | 1-22% |
| Kale | Brassica oleracea | Whole Leaf and stem | 1-22% |
| Green Cabbage | Brassica oleracea | Whole Head | 1-20% |
| Acerola | Malpighia emarginata | Fruit Juice | 1-20% |
| Asparagus | Asparagus officinalis | Whole Stalk | 1-20% |
| Brussel Sprouts | Brassica oleracea | Whole vegetable | 1-20% |
| Lime | Citrus xlatitifolia | Fruit Juice | 1-18% |
| Collards | Brassica oleracea | Whole leaf and steam | 1-18% |
| Romaine Lettuce | Lactuca sativa | Whole leaf and steam | 1-15% |
| Swiss Chard | Beta vulgaris ssp. cicla | Whole Vegetable | 1-15% |

TABLE 3
ORANGE/YELLOW PIGMENTED COMPOSITIONS

| Component | Latin Binomial | Part Used | Weight % |
|---|---|---|---|
| Carrot | Daucus carota | Whole Root/juice | 1-24% |
| Sweet Potato | Ipomoea batatas | Whole vegetable | 1-24% |
| Pumpkin | Cucurbita moschata | Whole vegetable | 1-24% |
| Butternut Squash | Cucurbita moschata | Whole vegetable | 1-23% |
| Banana | Musa acuminata | Whole fruit | 1-23% |
| Mango | Mangifera indica | Whole Fruit | 1-22% |
| Pineapple | Ananas comosus | Whole Fruit/juice | 1-21% |
| Acerola | Malpighia emarginata | Whole Fruit/Juice | 1-21% |
| Apricot | Prunus armeniaca | Whole fruit | 1-20% |
| Peach | Prunus persica | Whole fruit | 1-20% |
| Yellow Bell Pepper | Capsicum annuum | Whole fruit | 1-20% |
| Lemon | Citrus limon | Whole Fruit/juice | 1-18% |
| Orange | Citrus × sinensis | Whole Fruit/juice | 1-18% |

TABLE 4
PURPLE/BLUE PIGMENTED COMPOSITIONS

| Component | Latin Binomial | Part Used | Weight % |
|---|---|---|---|
| Beet | Beta vulgaris | Whole Root/juice | 1-24% |
| Purple Carrot | Daucus carota | Whole Root/juice | 1-24% |
| Concord Grape | Vitus labrusca | Whole Fruit/juice | 1-24% |
| Acai | Euterpe oleracea | Whole fruit | 1-24% |
| Blueberry | Vaccinium corymbosum | Whole fruit | 1-24% |
| Blackberry | Rubus fruticosus | Whole fruit | 1-22% |
| Purple cabbage | Brassica oleracea | Whole leaf and stem | 1-22% |
| Acerola | Malphighia emarginata | Whole Fruit/juice | 1-20% |
| Eggplant | Solanum melongena | Whole Vegetable | 1-20% |
| Plum | Prunus domestica | Whole fruit | 1-20% |
| Black Raspberry | Rubus leucodermis | Whole fruit | 1-20% |
| Black Currant | Ribes nigrum | Whole Fruit/juice | 1-18% |
| Passion Fruit | Passiflora edulis | Whole fruit | 1-18% |

TABLE 5
RED PIGMENTED COMPOSITIONS

| Component | Latin Binomial | Part Used | Weight % |
|---|---|---|---|
| Apple | Malus domestica | Whole Fruit | 1-24% |
| Beet | Beta vulgaris | Whole Root juice | 1-24% |
| Pear | Pyrus communis | Whole fruit | 1-24% |
| Strawberry | Fragaria × ananassa | Whole fruit | 1-24% |
| Raspberry | Rubus idaeus | Whole fruit | 1-20% |
| Cranberry | Vaccinium Macrocarpon | Whole fruit | 1-20% |
| Acerola | Malphighia emarginata | Whole Fruit/juice | 1-20% |
| Pomegranate | Punica granatum | Whole Fruit/juice | 1-20% |
| Red Peppers | Capsicum annuum | Whole fruit | 1-20% |
| Rhubarb | Rheum rhabarbarum | Whole fruit | 1-20% |
| Prickly Pear | Opuntia sp | Whole fruit | 1-20% |
| Tomato | Solanum lycopersicum | Whole vegetable | 1-20% |
| Cherry | Prunus cerasus | Fruit juice | 1-18% |

The components and/or weight percentages used in the compositions, such those shown above, can vary based on seasonal availability, harvest timing, and supply of particular vegetable, fruit or other agriculture-based sources. For example, if a particular component is unavailable due seasonal frost, another component from the same or another pigment source can be used as a replacement at the same or different weight percentage.

The foregoing description of illustrative examples has been presented for purposes of illustration and of description. It is not intended in be exhaustive or limiting with respect to the precise form disclosed, and modifications and variations are possible in light of the above teachings or may be acquired from practice of the disclosed aspects of the present invention. It is intended that the scope of the present invention be defined by the claims appended hereto and their equivalents.

1-29. (canceled)

30. A method for preparing a fruit and vegetable-based food product formulation for animal or human consumption, the method comprising:
providing fruit components and vegetable components, the fruit and vegetable components all being within a single pigment group selected from: green pigmented compositions, orange/yellow pigmented compositions, white pigmented compositions, black pigmented compositions, purple/blue pigmented compositions, and red pigmented compositions;
removing the fruit and vegetable components from direct sunlight and reducing respiration by hydro- or rapid-cooling of the fruit and vegetable components or maintaining a relative humidity of the fruit and vegetable components;
cleaning the fruit and vegetable components;
removing inedible parts of the fruit and vegetable components;
juicing, puréeing, and combining the fruit and vegetable components into a composition at a low temperature;
dehydrating the composition using a low-temperature dehydrating process;
testing the composition.

31. The method of claim 30, wherein the low-temperature dehydrating process comprises rapid zone drying.

32. The method of claim 30, wherein the low-temperature dehydrating process comprises window refractance drying.

33. The method of claim 30, wherein the low-temperature dehydrating process comprises vacuum belt drying.

34. The method of claim 30, wherein the low-temperature dehydrating process comprises floating an infrared-transparent on the surface of a body of heated water and placing the composition on top of the film to absorb heat from the water and thereby cause drying/dehydration of the composition.

35. The method of claim 34, wherein the composition is placed in an elongated trough reservoir on an endless strip of a polyester material floating on the surface of water in the reservoir.

36. The method of claim 30, wherein the low-temperature dehydrating process comprises:
placing the composition on a first side of a support surface;
directing dry infrared light toward a second side of the support surface to remove moisture from the composition, wherein the temperature of the infrared light is regulated as a function of at least one characteristic of the composition.

37. The method of claim 36, further comprising successively moving the composition through a plurality of control zones, each of the plurality of control zones having at least one associated light source and an associated sensor configured to regulate the temperature of the at least one associated light source independently of sensors associated with another control zone of the plurality of control zones.

38. The method of claim 30, further comprising harvesting the fruit and vegetable components at the time of ripening, crispness, freshness, taste, appearance, condition, and highest nutritive value.

39. The method of claim 30, further comprising blending the composition into a slurry.

40. The method of claim 30, wherein combining the composition comprises producing a powder, flakes, tablet, or capsule.

41. The method of claim 30, further comprising adding the composition to a cosmetic or topical-use product.

42. The method of claim 30, further comprising adding the composition to a food or supplement product for animal or human consumption.

43. The method of claim 30, further comprising packaging the composition in a pre-sterilized container or pre-sterilized package.

44. The method of claim 30, wherein at least 10 separate fruit and vegetable components are combined into the composition.

45. A method for preparing a fruit and vegetable-based food product formulation for animal or human consumption, the method comprising:
providing fruit components and vegetable components, the fruit and vegetable components all being within a single pigment group selected from: green pigmented compositions, orange/yellow pigmented compositions, white pigmented compositions, black pigmented compositions, purple/blue pigmented compositions, and red pigmented compositions, wherein the fruit and vegetable components are harvested at the time of ripening, crispness, freshness, taste, appearance, condition, and highest nutritive value;
removing the fruit and vegetable components from direct sunlight and reducing respiration by hydro- or rapid-cooling of the fruit and vegetable components or maintaining relative humidity of the fruit and vegetable components;
cleaning and preparing the fruit and vegetable components;
removing inedible parts of the fruit and vegetable components;
juicing, puréeing, and combining the fruit and vegetable components into a composition at a low temperature;
dehydrating the composition using a low-temperature dehydrating process, the low-temperature dehydrating process comprising at least one of rapid zone drying, window refractance drying, vacuum belt drying;
testing the composition;
blending the composition and a liquid into a slurry in a blender;
preparing the composition as a food or supplement product for animal or human consumption.

46. The method of claim 45, further comprising packaging the food or supplement product in a pre-sterilized container or pre-sterilized package.
